(19) United States
(12) Patent Application Publication
(10) Pub. No.: US 2025/0296905 A1
(43) Pub. Date: Sep. 25, 2025

(54) CARDANOL-BASED BISPHENOL, PREPARATION METHOD THEREFOR AND APPLICATION THEREOF

(71) Applicant: NASURFAR BIOMATERIAL TECHNOLOGY (CHANGSHU) CO., LTD., Suzhou (CN)

(72) Inventors: Ningzhong BAO, Suzhou (CN); Zhisheng DAI, Suzhou (CN); Liangyong CHU, Suzhou (CN); Lu XU, Suzhou (CN); Zhiwei ZHOU, Suzhou (CN)

(21) Appl. No.: 19/230,200

(22) Filed: Jun. 6, 2025

Related U.S. Application Data
(63) Continuation of application No. PCT/CN2023/134369, filed on Nov. 27, 2023.

(30) Foreign Application Priority Data
Dec. 9, 2022 (CN) 202211578375.7
Oct. 27, 2023 (CN) 202311407946.5

(51) Int. Cl. C07C 39/16; C07C 37/00; C07C 37/20; C07C 37/68; C07C 37/74; C07C 39/21; C08G 59/06; C08G 59/18; C09D 163/00

(52) U.S. Cl. CPC C07C 39/16; C07C 37/002; C07C 37/20; C07C 37/685; C07C 37/74; C07C 39/21; C08G 59/06; C08G 59/182; C09D 163/00

(57) ABSTRACT

A method for preparing bisphenol includes: mixing and uniformly stirring cardanol, formaldehyde and an alkaline catalyst, and reacting at a certain temperature to obtain a hydroxymethylation product A; washing the product A multiple times with water until the pH value is neutral, and then centrifuging to remove water to obtain a product B; mixing and uniformly stirring the product B with phenol and an acidic catalyst, and reacting at a certain temperature to obtain a product C after a phenolic alcohol reaction; washing the product C with water until neutral, and distilling same under reduced pressure to obtain a cardanol-based bisphenol product. Cardanol is used which has the structural characteristics of both a benzene ring and an alkane chain, and when protecting phenolic hydroxyl which has high reaction activity, a bisphenol structure which has both benzene ring rigidity and cardanol upper alkane long-chain toughness is obtained.

This application is a Continuation Application of PCT/CN2023/134369, filed on Nov. 27, 2023, which claims priority to Chinese Patent Application Nos. 202211578375.7, filed on Dec. 9, 2022, and 202311407946.5, filed on Oct. 27, 2023, all of which are incorporated by reference for all purposes as if fully set forth herein.

### TECHNICAL FIELD

The present disclosure belongs to the technical field of bisphenol materials and relates in particular to cardanol-based bisphenol, a preparation method therefor and application thereof.

### BACKGROUND OF THE INVENTION

Compared with monophenol, bisphenol (such as bisphenol A) usually has a plurality of benzene rings, rigidity of resin prepared thereby is greatly improved, and with the increase in a content of benzene rings, thermostability is greatly improved. Meanwhile, the bisphenol has two phenolic hydroxyls, which may provide additional active sites for improving reaction activity. Thus, the bisphenol materials have wide application prospects in the fields such as life sciences, composite materials and coatings.

At present, most of existing bisphenol is petroleum-based materials and has the defects of being non-renewable, having persistent reproduction toxicity and the like, and a small amount of natural bio-based bisphenol, such as cardol, has a low yield, is highly difficult to extract, and cannot meet market demands. Due to linear molecular chains and benzene ring rigidity of the existing petroleum-based bisphenol materials, prepared resin has poor fluidity and flexibility and high viscosity, and hardly meets demands of construction. Synthesis of a polyphenol material by using a bio-based cardanol material with a flexible carbon chain as a raw material is one of effective ways to solve the above problems.

Cardanol is a plant phenol product extracted from natural cashew nut shell liquid and usually replaces or partially replaces phenol for synthesis of epoxy resin, an epoxy curing agent, phenolic aldehyde resin and other materials. The cardanol further has many properties different from phenol while having molecular properties of phenol: it has a benzene ring structure, a larger molecular weight and resistance to a high temperature; phenolic hydroxyl on a benzene ring may provide wettability and activity of a system to a contact surface; a carbon-15 straight chain with an unsaturated double bond at a meta-position on the benzene ring may provide good toughness of the system, excellent hydrophobicity, low permeability and self-dryness, which is regarded as an ideal biomass raw material. Methods for synthesis of bisphenol by using cardanol as a raw material reported in current literature mainly use a carbon-carbon double bond in a cardanol carbon-15 chain to obtain a bisphenol material with phenol and other monophenol materials for addition. However, since a variety of carbon-carbon double bonds exist in a cardanol R chain and have different activities, the bisphenol material has the problems of uncontrollable structure, poor component singularity and low yield. Moreover, in a bisphenol molecule generated by a method of R chain carbon-carbon double bond addition, the R chain participates in crosslinking, consequently, an effect of improving toughness is lost, the viscosity and toughness of resin are greatly reduced, and thus this type of materials cannot replace application of the petroleum-based materials.

Based on the above problems in the prior art, the present disclosure is provided.

### SUMMARY OF THE INVENTION

To overcome the defects in the prior art, the present disclosure provides cardanol-based bisphenol, a preparation method therefor and application thereof. The present disclosure obtains a cardanol-based bisphenol structure having a high yield, a soft alkane long chain and a rigid benzene ring at the same time and a high-activity site through reactions in simple experiment steps. The resin material has both rigidity and toughness, as well as low viscosity and high toughness, and may greatly widen application of a cardanol bio-based material in the fields such as composite materials, life sciences, surfactants and friction powder.

A technical solution of the present disclosure is as follows.

The present disclosure relates to cardanol-based bisphenol, where a structure is shown as follows:

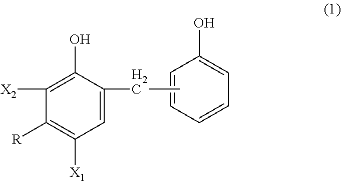

where a group R is C₁₅H₃₁₋₂ₙ, where n=0-3,

in a case of n=0, C₁₅H₃₁ is

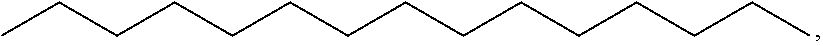

in a case of n=1, C₁₅H₂₉ is

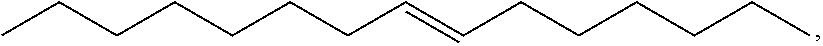

in a case of n=2, C₁₅H₂₇ is

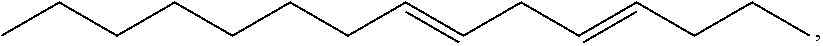

and

in a case of n=3, C₁₅H₂₅ is

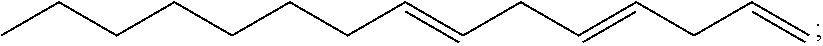

and

in Formula (1), a linking group between two benzene rings is methylene, and the methylene is located at an ortho-position or a para-position of phenolic hydroxyl on the benzene ring on the right side.

Preferably, groups X₁ and X₂ are the same and are H or CH₂OH.

The Present Disclosure Further Relates to a Method for Preparing Cardanol-Based Bisphenol, Including the Following Steps:
(1) mixing and uniformly stirring cardanol, formaldehyde and an alkaline catalyst first, and reacting at a certain temperature to obtain a hydroxymethylation product A;
(2) washing the product A multiple times with water until a pH value is neutral, and then centrifuging to remove water to obtain a product B, where this step removes unreacted formaldehyde, alkaline catalyst and water;
(3) mixing and uniformly stirring the product B with excess phenol and an acidic catalyst, and reacting at a certain temperature to obtain a product C after a phenolic alcohol reaction; and
(4) washing the product C with water until neutral, and distilling same under reduced pressure to obtain a cardanol-based bisphenol product, where this step removes impurities in the product.

A synthesis mechanism involved in the preparation method is as follows:

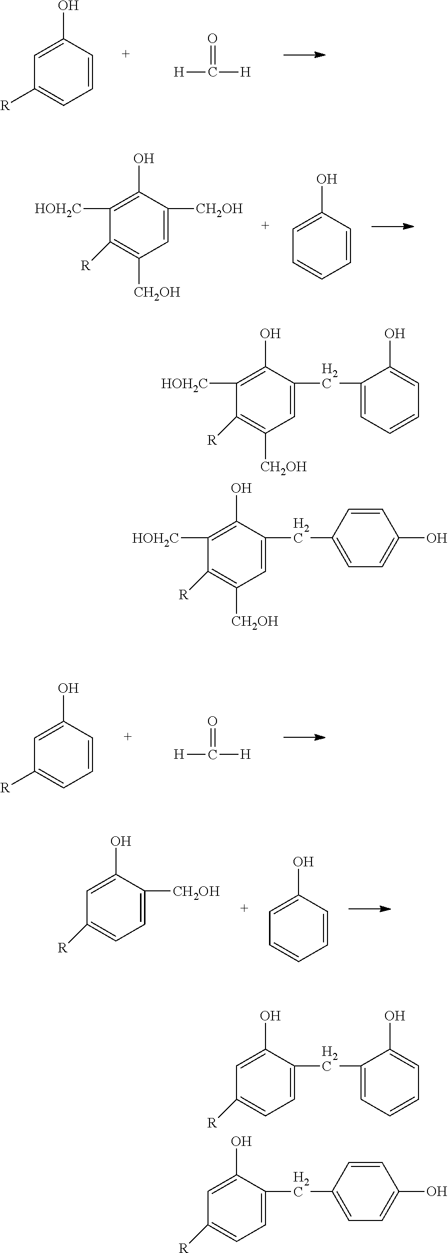

The raw material cardanol in the preparation method is a bio-based resource without occupying grain and petroleum resources and is intended to replace application of a petroleum-based material in the related art. According to the present disclosure, the structural characteristics of having both benzene rings and an alkane chain of the cardanol are utilized, and on the premise of protecting the phenolic hydroxyl which has high reaction activity, a bisphenol structure which has both benzene ring rigidity and alkane long-chain toughness on the cardanol is obtained.

Preferably, in step (1), a molar ratio of the cardanol to the formaldehyde is 1:1 to 1:5, and a mass ratio of the cardanol to the alkaline catalyst is 1:0.001 to 1:0.05.

Preferably, in step (1), the reaction is performed for 1 h to 7 h at a temperature ranging from 30° C. to 90° C.

Preferably, in step (1), the alkaline catalyst is at least one of ammonium hydroxide, triethylamine, barium hydroxide, sodium hydroxide or magnesium hydroxide. Further preferably, the alkaline catalyst is at least one of ammonium hydroxide, triethylamine or magnesium hydroxide.

Preferably, a molar ratio of the cardanol to the phenol in step (3) is 1:1 to 1:12; and a use amount of the acidic catalyst in step (3) is 0.001 to 0.05 of the mass of the cardanol.

Preferably, in step (3), the reaction is performed for 1 h to 7 h at a temperature ranging from 40° C. to 120° C.

Preferably, in step (3), the acidic catalyst is at least one of oxalic acid, phosphoric acid, hydrochloric acid, sulfuric acid, dodecylbenzene sulfonic acid, p-hydroxybenzenesulfonic acid or p-toluenesulfonic acid monohydrate.

Preferably, in step (4), distilling under reduced pressure is performed for 1 h to 7 h under a pressure of 0.1 MPa at a temperature ranging from 60° C. to 110° C.

The present disclosure further relates to a method for preparing cardanol phenol based epoxy resin, which uses cardanol-based bisphenol as a raw material to react with epoxy chloropropane for epoxidation to generate cardanol-based bisphenol epoxy resin. The method includes the following steps:
(1) stirring the cardanol-based bisphenol and the epoxy chloropropane as raw materials for 5 min to 15 min at 50° C. to 70° C. in the presence of a quaternary ammonium salt catalyst, then adding a NaOH solution, and maintaining a constant temperature for 40 min to 90 min;
(2) raising a temperature to 75° C., further adding a NaOH solution, maintaining reflux with water separation, testing a recovered water amount, stopping a reaction after the recovered water amount reaches a theoretical amount, and performing distilling under reduced pressure to remove the epoxy chloropropane;
(3) filtering or centrifuging to remove a salt; and
(4) obtaining a filtrate, raising a temperature to 110° C., further performing distilling under reduced pressure to remove the epoxy chloropropane, and discharging to obtain the cardanol phenol based epoxy resin.

Taking

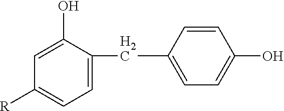

as an example, a synthesis mechanism of the cardanol phenol based epoxy resin is as follows:

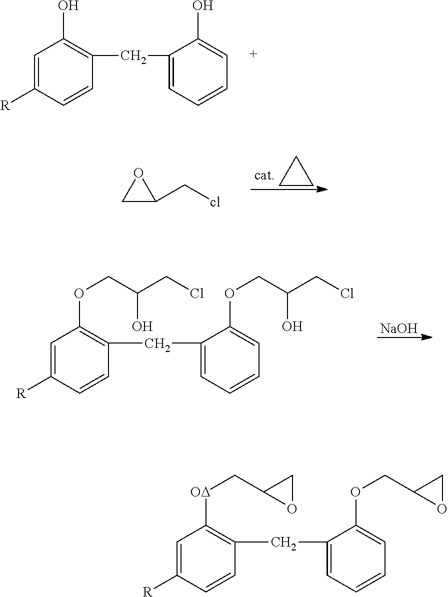

Preferably, a mass concentration of the NaOH solution used in step (1) and step (2) is 30% to 60%.

Preferably, the quaternary ammonium salt catalyst is added in a form of a solution and is tetraethylammonium bromide.

Preferably, in step (2), distilling under reduced pressure to remove the epoxy chloropropane takes 1 h to 3 h; and in step (4), further distilling under reduced pressure to remove the epoxy chloropropane takes 1 h to 3 h.

The present disclosure further relates to cardanol phenol based epoxy resin, prepared by the above preparation method.

A method for preparing an anticorrosive coating based on cardanol phenol based epoxy resin includes the following steps:
(1) obtaining a product a by mixing, heating and uniformly stirring the cardanol phenol based epoxy resin and a curing agent according to a certain proportion, where mixing is performed for 5 min to 15 min at a temperature ranging from 20° C. to 50° C.; where the curing agent is an amido curing agent;
(2) performing ultrasound on the product a for several minutes, then putting the product a in a vacuum drying oven for vacuumizing and defoaming to obtain a product b; and
(3) knife-coating the product b onto a substrate, and performing curing in a 30° C. to 90° C. drying oven to obtain a final product.

The present disclosure has the following beneficial effects.
(1) The present disclosure uses cardanol, the formaldehyde and the phenol as the raw materials for synthesis of the novel bisphenol material, the material achieves double benzene rings of a molecule, thus improves molecular rigidity and thermostability and meanwhile maintains a freedom degree of an R chain, thus a resin material prepared from the bisphenol material has low viscosity and high toughness; and the novel bio-based bisphenol material has a molecular structure having both high strength and high toughness, is expected to replace application of a petroleum-based bisphenol material in fields such as composite materials, coatings, surfactants, friction powder and life medicine and has great application prospects.
(2) The preparation method of the present disclosure is simple and reliable, a bisphenol molecule having both benzene ring rigidity and alkane long-chain toughness on cardanol can be obtained, the purity of the prepared product may reach 85%, and the bisphenol is environmentally friendly and low in toxicity and complies with requirements of environmental protection and sustainable development.
(3) The epoxy coating is prepared through two steps by using the cardanol-based bisphenol, firstly, the cardanol-based bisphenol is used as a raw material for preparing the cardanol phenol based epoxy resin, then the cardanol phenol based epoxy resin is used for preparing a bio-based shape memory epoxy resin coating, and the coating has a high crosslinking density, good hydrophobicity and good corrosion resistance and further has excellent mechanical properties, including high toughness, high adhesion and impact resistance.

### BRIEF DESCRIPTION OF DRAWINGS

The present disclosure is further described below with reference to the accompanying drawings and examples.

FIG. 1 is a gas chromatography-mass spectrometry spectrum of a cardanol raw material and a partially-enlarged view thereof.

FIG. 2 is a gas chromatogram of a hydroxymethylation intermediate product of a phenolic aldehyde reaction product.

FIG. 3 is a gas chromatography-mass spectrometry spectrum of a hydroxymethylation intermediate product of a phenolic aldehyde reaction product.

FIG. 4 is a gas chromatogram of a cardanol-based bisphenol product of a phenolic alcohol reaction product.

FIG. 5 is a first gas chromatography-mass spectrometry spectrum of a cardanol-based bisphenol product of a phenolic alcohol reaction product.

FIG. 6 is a second gas chromatography-mass spectrometry spectrum of a cardanol-based bisphenol product of a phenolic alcohol reaction product.

FIG. 7 is a third gas chromatography-mass spectrometry spectrum of a cardanol-based bisphenol product of a phenolic alcohol reaction product.

FIG. 8 is a fourth gas chromatography-mass spectrometry spectrum of a cardanol-based bisphenol product of a phenolic alcohol reaction product.

FIG. 9 is a nuclear magnetic resonance hydrogen spectrum of a cardanol-based bisphenol product of a phenolic alcohol reaction product.

FIG. 10 is a nuclear magnetic resonance carbon spectrum of a cardanol-based bisphenol product of a phenolic alcohol reaction product.

FIG. 11 is a Fourier transform infrared spectrum of cardanol-based bisphenol and cardanol phenol based epoxy resin.

FIG. 12 is an electrochemical spectrum of a coating prepared by using cardanol phenol based epoxy resin.

FIG. 13 is a synthesis flowchart of cardanol phenol based epoxy resin.

FIG. 14 is a flowchart of preparing an epoxy coating by using cardanol phenol based epoxy resin.

### DETAILED DESCRIPTION OF THE INVENTION

To make the objectives, technical solutions, and advantages of the present disclosure clearer, the following further describes the present disclosure in detail with reference to the accompanying drawings and specific implementations. It is to be understood that these descriptions are merely exemplary instead of limiting the scope of the present disclosure. Besides, in the following description, description of known structures and technologies is omitted to avoid unnecessary confusing of the concepts of the present disclosure.

### Example 1

105.312 g of cardanol, 0.105 g (containing 25% to 28% of ammonia) of ammonium hydroxide and 11.444 g of paraformaldehyde were taken and added into a 500 mL four-necked flask, a temperature was increased to 30° C., and then a reaction was performed for 1 h; then washing was performed multiple times with water until a pH value was neutral, and then centrifuging was performed to remove water; then a temperature was increased to 40° C., and 0.105 g (0.00117 mol) of oxalic acid and 33.033 g (0.351 mol) of phenol were further added for performing a phenolic alcohol condensation reaction for 1 h; then washing was performed multiple times with water until a pH value was neutral; and then distilling under reduced pressure was performed for 1 h at 60° C. under a pressure of 0.1 MPa to obtain a product (the purity of cardanol-based bisphenol was 60%).

### Example 2

46.182 g of cardanol, 0.924 g (0.00913 mol) of triethylamine and 15.055 g of paraformaldehyde were taken and added into a 500 mL four-necked flask, a temperature was increased to 60° C., and then a reaction was performed for 3 h; then washing was performed multiple times with water until a pH value was neutral, and then centrifuging was performed to remove water; then a temperature was increased to 100° C., and 0.924 g (0.00943 mol) of phosphoric acid and 86.915 g (0.924 mol) of phenol were further added for performing a phenolic alcohol condensation reaction for 3 h; then washing was performed multiple times with water until a pH value was neutral; and then distilling under reduced pressure was performed for 3 h at 90° C. under a pressure of 0.1 MPa to obtain a product (the purity of cardanol-based bisphenol was 67%).

### Example 3

27.740 g of cardanol, 1.387 g (0.0347 mol) of sodium hydroxide and 15.072 g of paraformaldehyde were taken and added into a 500 mL four-necked flask, a temperature was increased to 90° C., and then a reaction was performed for 7 h; then washing was performed multiple times with water until a pH value was neutral, and then centrifuging was performed to remove water; then a temperature was increased to 160° C., and 1.387 g (0.00425 mol) of dodecylbenzene sulfonic acid and 104.414 g (1.110 mol) of phenol were further added for performing a phenolic alcohol condensation reaction for 7 h; then washing was performed multiple times with water until a pH value was neutral; and then distilling under reduced pressure was performed for 6 h at 110° C. under a pressure of 0.1 MPa to obtain a product (the purity of cardanol-based bisphenol was 70%).

### Example 4

46.182 g of cardanol, 0.924 g (0.00913 mol) of triethylamine and 15.055 g of paraformaldehyde were taken and added into a 500 mL four-necked flask, a temperature was increased to 60° C., and then a reaction was performed for 3 h; then washing was performed multiple times with water until a pH value was neutral, and then centrifuging was performed to remove water; then a temperature was increased to 100° C., and 0.924 g (0.00943 mol) of sulfuric acid and 86.915 g (0.924 mol) of phenol were further added for performing a phenolic alcohol condensation reaction for 3 h; then washing was performed multiple times with water until a pH value was neutral; and then distilling under reduced pressure was performed for 3 h at 90° C. under a pressure of 0.1 MPa to obtain a product (the purity of cardanol-based bisphenol was 79%).

### Example 5

46.182 g of cardanol, 0.924 g (0.00539 mol) of barium hydroxide and 15.055 g of paraformaldehyde were taken and added into a 500 mL four-necked flask, a temperature was increased to 60° C., and then a reaction was performed for 3 h; then washing was performed multiple times with water until a pH value was neutral, and then centrifuging was performed to remove water; then a temperature was increased to 100° C., and 0.924 g (0.00943 mol) of sulfuric acid and 86.915 g (0.924 mol) of phenol were further added for performing a phenolic alcohol condensation reaction for 3 h; then washing was performed multiple times with water until a pH value was neutral; and then distilling under reduced pressure was performed for 3 h at 90° C. under a pressure of 0.1 MPa to obtain a product (the purity of cardanol-based bisphenol was 85%).

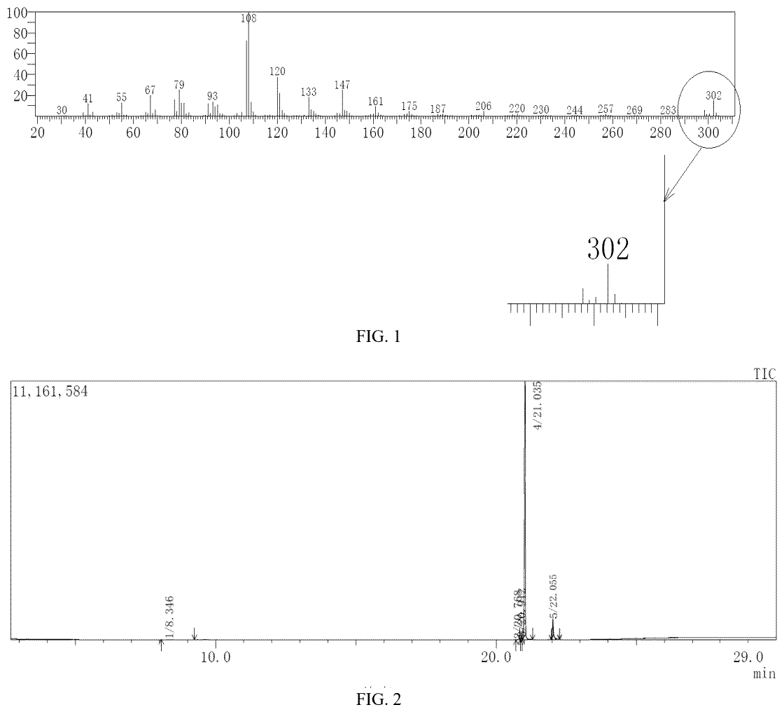

FIG. 1 is a gas chromatography-mass spectrometry spectrum of a cardanol raw material used in Examples 1 to 5 and a partially-enlarged view thereof. By enlarging a peak nearby 302, it can be seen that substance 298, 300, 302 and 304 correspond respectively to n=3, 2, 1, and 0, which indicates that the cardanol raw material itself is a mixture. In addition, it can also be seen from FIG. 1 that many peaks marked with reference numerals are not single peaks, and there are other peaks nearby, which further indicates that the cardanol raw material is a mixture.

Structures of cardanol-based bisphenol resin samples obtained in Examples 1 to 5 are determined, where a specific detection method is as follows: whether a related framework of a new structure conforms to a conception is characterized by using a nuclear magnetic resonance hydrogen spectrum and carbon spectrum, and gas chromatography-mass spectrometry (GCMS) is used for corresponding to a unique molecular weight structure to prove an intermediate product and a product structure. Taking Example 5 as an example, a corresponding spectrum is as follows.

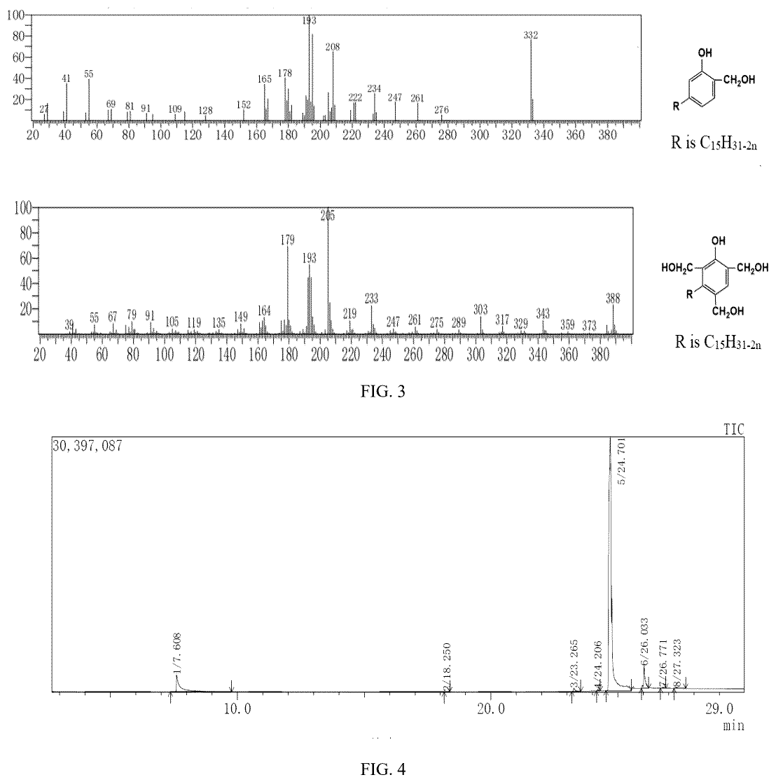

FIG. 2 is a gas chromatogram of a hydroxymethylation intermediate product of a phenolic aldehyde reaction product. FIG. 3 is two gas chromatography-mass spectrometry spectrums of the hydroxymethylation intermediate product of the phenolic aldehyde reaction product. In the gas chromatogram corresponding to GC-MS, substance appearing at 21.035 min corresponds to the first spectrum in FIG. 3 with a content of 92.43%, which represents a monohydroxymethylation intermediate product. In the gas chromatogram corresponding to GC-MS, a peak appearing at 22.055 min corresponds to the second spectrum in FIG. 3 with a content of 5.54%, which represents a trihydroxymethylation intermediate product. Other peaks in the gas chromatogram corresponding to GC-MS further include unreacted cardanol and impurities in a solvent, which have a quite small content. It can be known from the GCMS spectrum that an addition reaction of the formaldehyde and cardanol is successfully performed, only two products are generated in addition to the mass spectrum of the cardanol, it is determined that the two products are intermediates of a target product, and structural formulas of the two intermediates are as shown in FIG. 3.

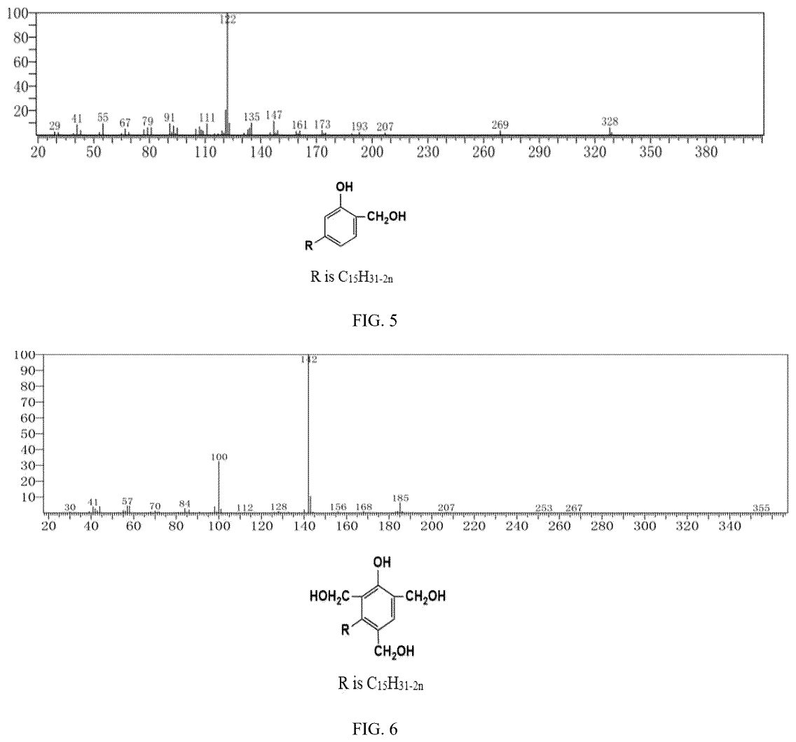

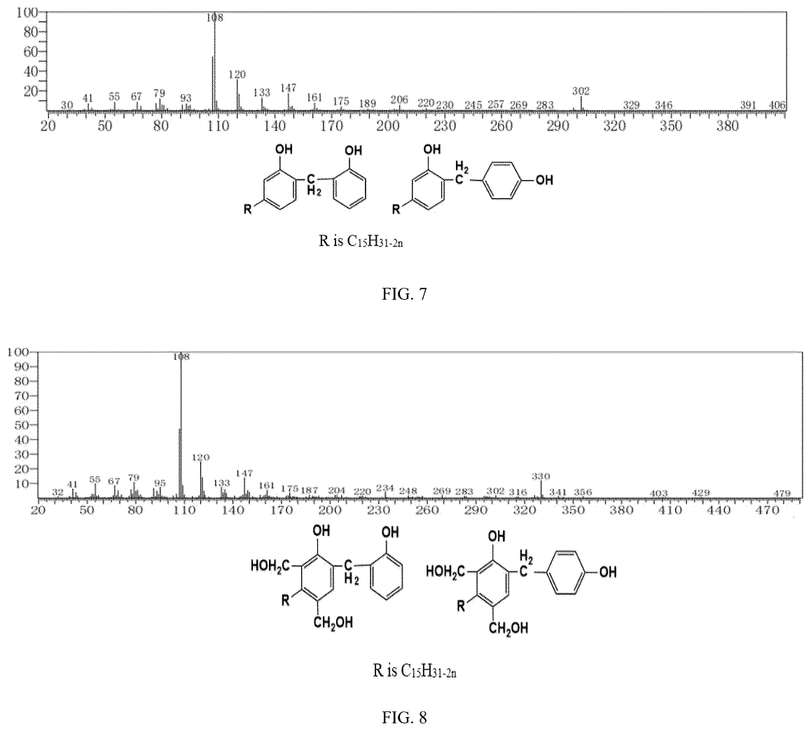

FIG. 4 is a gas chromatogram of a cardanol-based bisphenol product of a phenolic alcohol reaction product. FIG. 5 to FIG. 8 are gas chromatography-mass spectrometry spectrums of the cardanol-based bisphenol product of the phenolic alcohol reaction product. In the gas chromatogram corresponding to GC-MS, substance at 7.608 min is unreacted phenol, and peaks of substance at 23.265 min and 24.206 min correspond to the spectrums of FIG. 5 and FIG. 6. In the gas chromatogram corresponding to GC-MS, substance at 24.701 min corresponds to the spectrum of FIG. 7, and is a main product accounting for a large proportion, which is inferred to be bisphenol converted from a monohydroxymethylation product. In the gas chromatogram corresponding to GC-MS, a peak at 26.033 min corresponds to the spectrum of FIG. 8, which is inferred to be bisphenol converted from a trihydroxymethylation product. It can be known according to the GCMS spectrum that the product contains the bisphenol converted from the monohydroxymethylation product and the bisphenol converted from the trihydroxymethylation product.

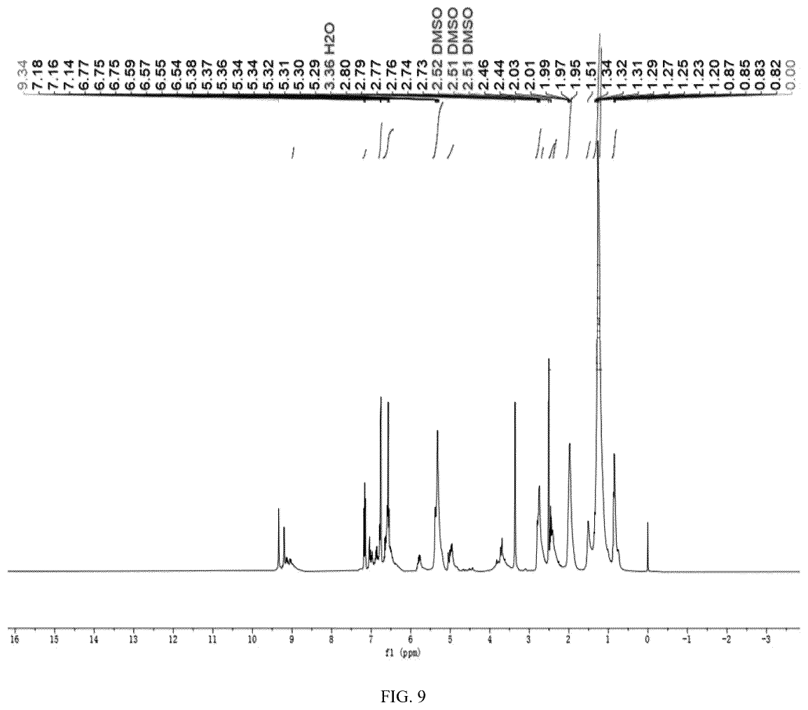

FIG. 9 is a nuclear magnetic resonance hydrogen spectrum of a cardanol-based bisphenol product of a phenolic alcohol reaction product. According to a 1H NMR spectrum of a sample, chemical shift corresponding to hydrogen of methylene is 3.5 ppm to 4.5 ppm, which determines the presence of methylene.

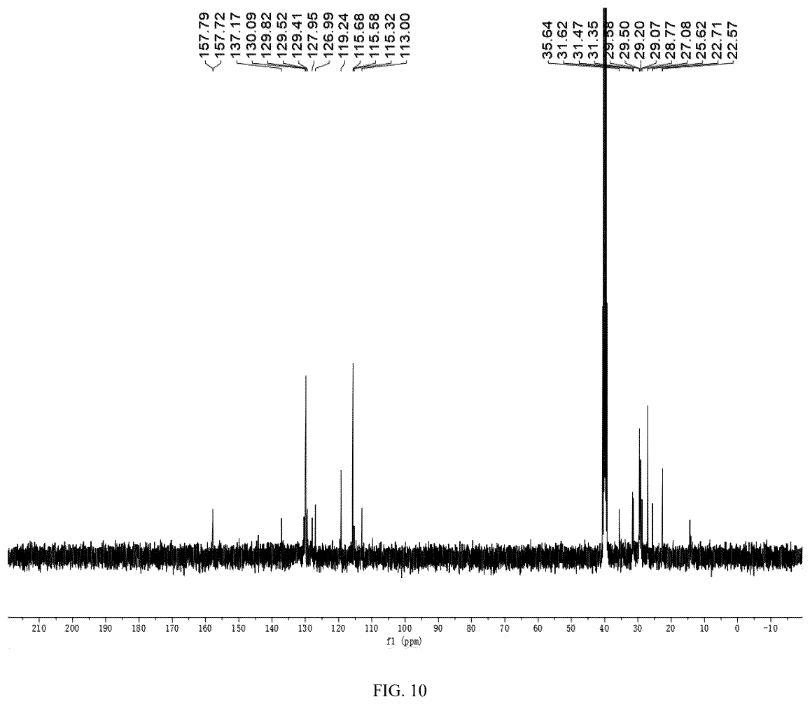

FIG. 10 is a nuclear magnetic resonance carbon spectrum of the cardanol-based bisphenol product of the phenolic alcohol reaction product. According to 12C NMR of a sample, chemical shift of C of 27-32 ppm of C—CH₂—C can be observed, and it can be judged in combination with the C spectrum and the H spectrum that a desired target product structure exists in the product.

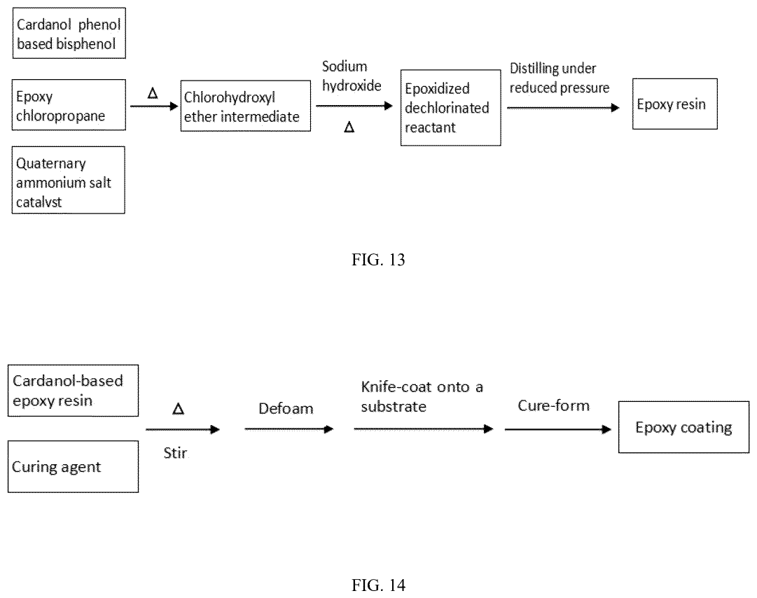

Cardanol phenol based bisphenol is prepared with reference to the method in Example 5, epoxy resin is prepared by using the cardanol phenol based bisphenol as a raw material, and specific flows are as shown in FIG. 13.

### Example 6 Synthesis of Epoxy Resin

(1) Etherification: cardanol-based bisphenol (50.7 g), epoxy chloropropane (55.2 g), and tetraethylammonium bromide (0.063 g, a catalyst being dissolved with 0.063 g of deionized water) were weighed, added into a four-necked flask, and stirred for 10 min at 50° C., then 4 g of a 33 wt. % NaOH solution was added, and a constant temperature was maintained for 40 min.

(2) Ring closure: a temperature was raised to 75° C., 22.533 g of a 33 wt. % NaOH solution was further dropwise added, reflux with water separation was maintained, a recovered water amount was tested, a reaction was stopped after the recovered water amount reached a theoretical amount, and distilling under reduced pressure was performed to remove the epoxy chloropropane for 1 h.

(3) Filtering was performed to remove a salt.

(4) A filtrate was taken, the temperature was raised to 110° C., distilling under reduced pressure was further performed to remove the epoxy chloropropane for 1 h, and discharging was performed to obtain a product.

### Example 7 Synthesis of Epoxy Resin

(1) Etherification: cardanol-based bisphenol (50.7 g), epoxy chloropropane (73.6 g), and tetraethylammonium bromide (0.084 g, a catalyst being dissolved with deionized water of the equivalent mass) were weighed, added into a four-necked flask, and stirred for 10 min at 60° C., then 6 g of a 33 wt. % NaOH solution was added, and a constant temperature was maintained for 50 min.

(2) Ring closure: a temperature was raised to 75° C., 33.8 g of a 33 wt. % NaOH solution was further dropwise added, reflux with water separation was maintained, a recovered water amount was tested, a reaction was stopped after the recovered water amount reached a theoretical amount, and distilling under reduced pressure was performed to remove the epoxy chloropropane for 2 h.

(3) Filtering was performed to remove a salt.

(4) A filtrate was taken, the temperature was raised to 110° C., distilling under reduced pressure was further performed to remove the epoxy chloropropane for 1 h, and discharging was performed to obtain a product.

### Example 8 Synthesis of Epoxy Resin

(1) Etherification: cardanol-based polyphenol (50.7 g), epoxy chloropropane (82.8 g), and tetraethylammonium bromide (0.105 g, a catalyst being dissolved with deionized water of the equivalent mass) were weighed, added into a four-necked flask, and stirred for 10 min at 70° C., then 8 g of a 33 wt. % NaOH solution was added, and a constant temperature was maintained for 60 min.

(2) Ring closure: a temperature was raised to 75° C., 45.066 g of a 33 wt. % NaOH solution was further dropwise added, reflux with water separation was maintained, a recovered water amount was tested, a reaction was stopped after the recovered water amount reached a theoretical amount, and distilling under reduced pressure was performed to remove the epoxy chloropropane for 3 h.

(3) Filtering was performed to remove a salt.

(4) A filtrate was taken, the temperature was raised to 110° C., distilling under reduced pressure was further performed to remove the epoxy chloropropane for 1 h, and then discharging was performed to obtain a product.

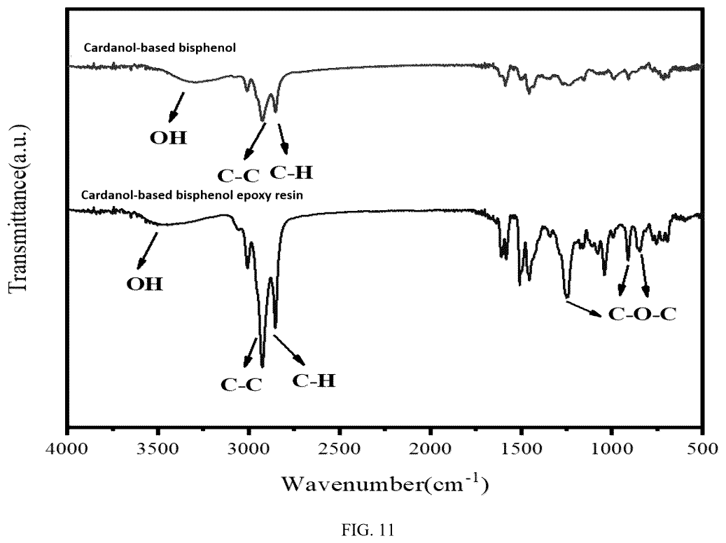

The cardanol-based bisphenol prepared by the method in Example 5 and the cardanol phenol based epoxy resin prepared in Example 7 are characterized, corresponding Fourier transform infrared spectrums are as shown in FIG. 11, and characteristic peaks of the cardanol-based bisphenol appear at peaks 3325 cm⁻¹, 2925 cm⁻¹ and 2843 cm⁻¹, which correspond respectively to tensile vibration of —OH, —C—C and —C—H. In addition, it can be discovered that after epoxidation, strong epoxy group characteristic peaks appear, namely, peaks at 1250 cm⁻¹, 910 cm⁻¹ and 770 cm⁻¹. Thus, it may indicate that the bisphenol is successfully epoxidized.

The cardanol phenol based epoxy resin (EP) is prepared with reference to the method in Example 6. According to the flow shown in FIG. 14, a coating is prepared by using the cardanol phenol based epoxy resin, and performance of the coating product is tested, specifically as follows.

### Example 9 Preparation of a Coating

10 g of cardanol phenol based epoxy resin and 3.7 g of a curing agent 718A were taken and added into a 100 mL flask, a temperature was raised to 30° C. and then mixing and stirring were performed for 15 min; a mixture was further placed in an ultrasonic machine to be subjected to ultrasonic processing for 10 min, and then placed in a vacuum drying oven for vacuum defoaming; and then knife-coating of a product onto a steel plate with a thickness of 100 μm was further performed, and then the knife-coated steel plate was placed in a 30° C. oven to be cured for 12 h to obtain a coating product.

### Example 10 Preparation of a Coating

10 g of cardanol phenol based epoxy resin and 3.7 g of a curing agent 718A were taken and added into a 100 mL flask, a temperature was raised to 40° C. and then mixing and stirring were performed for 10 min; a mixture was further placed in an ultrasonic machine to be subjected to ultrasonic processing for 10 min, and then placed in a vacuum drying oven for vacuum defoaming; and then knife-coating of a product onto a steel plate with a thickness of 100 μm was further performed, and then the knife-coated steel plate was placed in a 60° C. oven to be cured for 12 h to obtain a coating product.

### Example 11 Preparation of a Coating

10 g of cardanol phenol based epoxy resin and 3.7 g of a curing agent (PLR718A, NASURFAR biomaterial technology (Changshu) Co., Ltd.) were taken and added into a 100 mL flask, a temperature was raised to 50° C. and then mixing and stirring were performed for 5 min; a mixture was further placed in an ultrasonic machine to be subjected to ultrasonic processing for 10 min, and then placed in a vacuum drying oven for vacuum defoaming; and then knife-coating of a product onto a steel plate with a thickness of 100 μm was further performed, and then the knife-coated steel plate was placed in a 90° C. oven to be cured for 12 h to obtain a coating product.

### Comparative Example 1 Preparation of a Coating

10 g of petroleum-based epoxy resin E51, 3.7 g of a curing agent (PLR718A, NASURFAR biomaterial technology (Changshu) Co., Ltd.) and 0.5 g of acetone were taken and added into a 100 mL flask, a temperature was raised to 30° C. and then mixing and stirring were performed for 10 min; a mixture was further placed in an ultrasonic machine to be subjected to ultrasonic processing for 10 min, and then placed in a vacuum drying oven for vacuum defoaming; and then knife-coating of a product onto a steel plate with a thickness of 100 μm was further performed, and then the knife-coated steel plate was placed in a 30° C. oven to be cured for 12 h to obtain a coating product.

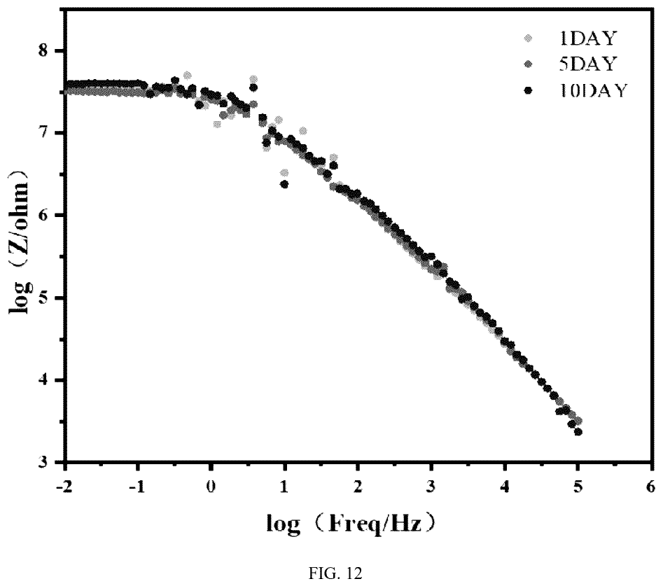

FIG. 12 is an electrochemical spectrum of the coating prepared from the cardanol phenol based epoxy resin in Example 11. After a 10-day test, under a test condition of 0.01 Hz, impedance of a steel plate substrate coated with the coating is 10\hat{ }7.5. It can be seen according to the electrochemical spectrum that impedance performance of the anticorrosive coating is high, which proves that the coating has good corrosion resistance.

Mechanical properties of the coating prepared in Example 11 and the coating prepared in Comparative example 1 are tested, and specific comparison results of the mechanical properties are shown in Table 1.

TABLE 1

| | Hardness | Adhesion | Impact | Flexibility/ |
|---|---|---|---|---|
| | property | property | resistance | mm |
| EP + 718A | H | Grade 1 | Positive/cm | Grade 1 |
| (curing agent) | | | 110 | |
| | | | Negative/cm 80 | |
| E51 + acetone + | 2H | Grade 5 | Positive/cm 10 | Grade 1 |
| 718A | | | Negative/cm 5 | |

It can be seen from Table 1 that compared with the petroleum-based epoxy resin (E51) coating, adhesion, flexibility and impact resistance of the coating prepared from the cardanol phenol based epoxy resin (EP) are greatly improved.

It is to be understood that the above specific implementations of the present disclosure are merely for exemplary descriptions or for explaining principles of the present disclosure instead of limiting the present disclosure. Thus, any modification, equivalent replacement and improvement made without departing from the spirit and scope of the present disclosure shall fall within the protection scope of the present disclosure. In addition, the appended claims of the present disclosure are intended to cover all variations and modified examples falling within the scope and the boundary of the appended claims, or within equivalent forms of such scope and boundary.

1. Cardanol-based bisphenol, having Formula (1): 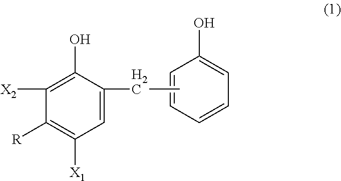
wherein a group R is C₁₅H₃₁₋₂ₙ, wherein n=0-3,
in a case of n=0, C₁₅H₃₁ is 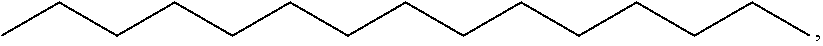
in a case of n=1, C₁₅H₂₉ is 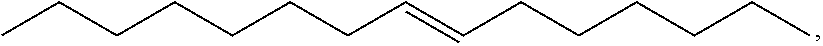
in a case of n=2, C₁₅H₂₇ is 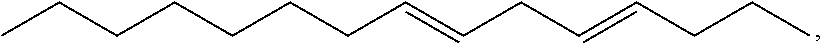
and
in a case of n=3, C₁₅H₂₅ is 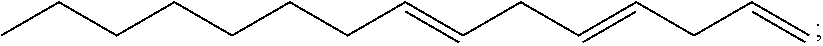
in Formula (1), a linking group between two benzene rings is methylene, and the methylene is located at an ortho-position or a para-position of phenolic hydroxyl on the benzene ring on the right side; and
groups X₁ and X₂ are the same and are H or CH₂OH.

2. A method for preparing cardanol-based bisphenol, comprising the following steps:
(1) mixing and uniformly stirring cardanol, formaldehyde and an alkaline catalyst first, and reacting at a certain temperature to obtain a hydroxymethylation product A;
(2) washing the product A multiple times with water until a pH value is neutral, and then centrifuging to remove water to obtain a product B;
(3) mixing and uniformly stirring the product B with phenol and an acidic catalyst, and reacting at a certain temperature to obtain a product C after a phenolic alcohol reaction; and
(4) washing the product C with water until neutral, and distilling same under reduced pressure to obtain a cardanol-based bisphenol product.

3. The method for preparing the cardanol-based bisphenol according to claim 2, wherein in step (1), a molar ratio of the cardanol to the formaldehyde is 1:1 to 1:5, and a mass ratio of the cardanol to the alkaline catalyst is 1:0.001 to 1:0.05.

4. The method for preparing the cardanol-based bisphenol according to claim 2, wherein in step (1), the reaction is performed for 1 h to 7 h at a temperature ranging from 30° C. to 90° C.

5. The method for preparing the cardanol-based bisphenol according to claim 2, wherein in step (1), the alkaline catalyst is at least one of ammonium hydroxide, triethylamine, barium hydroxide, sodium hydroxide or magnesium hydroxide.

6. The method for preparing the cardanol-based bisphenol according to claim 2, wherein a molar ratio of the cardanol to the phenol in step (3) is 1:1 to 1:12; and a use amount of the acidic catalyst in step (3) is 0.001 to 0.05 of mass of the cardanol.

7. The method for preparing the cardanol-based bisphenol according to claim 2, wherein in step (3), the reaction is performed for 1 h to 7 h at a temperature ranging from 40° C. to 120° C.

8. The method for preparing the cardanol-based bisphenol according to claim 2, wherein in step (3), the acidic catalyst is at least one of oxalic acid, phosphoric acid, hydrochloric acid, sulfuric acid, dodecylbenzene sulfonic acid, p-hydroxybenzenesulfonic acid or p-toluenesulfonic acid monohydrate.

9. The method for preparing the cardanol-based bisphenol according to claim 2, wherein in step (4), distilling under reduced pressure is performed for 1 h to 7 h under a pressure of 0.1 MPa at a temperature ranging from 60° C. to 110° C.

10. A method for preparing a cardanol phenol based epoxy resin, wherein by using the cardanol-based bisphenol according to claim 1 as raw material, the method comprises the following steps:
(1) stirring the cardanol-based bisphenol and epoxy chloropropane as raw materials for 5 min to 15 min at 50° C. to 70° C. in the presence of a quaternary ammonium salt catalyst, then adding a NaOH solution, and maintaining a constant temperature for 40 min to 90 min;
(2) raising a temperature to 75° C., further adding a NaOH solution, maintaining reflux with water separation, testing a recovered water amount, stopping a reaction after the recovered water amount reaches a theoretical amount, and performing distilling under reduced pressure to remove the epoxy chloropropane;
(3) filtering or centrifuging to remove a salt; and
(4) obtaining a filtrate, raising a temperature to 110° C., further performing distilling under reduced pressure to remove the epoxy chloropropane, and discharging to obtain the cardanol phenol based epoxy resin.

11. The cardanol phenol based epoxy resin, prepared by the preparation method according to claim 10.

12. A method for preparing an anticorrosive coating based on the cardanol phenol based epoxy resin according to claim 11, comprising the following steps:
(1) obtaining a product a by mixing, heating and uniformly stirring the cardanol phenol based epoxy resin and a curing agent according to a certain proportion, wherein mixing is performed for 5 min to 15 min at a temperature ranging from 20° C. to 50° C.;
(2) performing ultrasound on the product for several minutes, then putting the product a in a vacuum drying oven for vacuumizing and defoaming to obtain a product b; and
(3) knife-coating the product b onto a substrate, and performing curing in a 30° C. to 90° C. drying oven to obtain a final product.
